(19) United States
(12) Patent Application Publication
(10) Pub. No.: US 2006/0089319 A1
(43) Pub. Date: Apr. 27, 2006

(54) Method for treatment and prevention of bacterial vaginosis

(76) Inventor: Dean N. Pollack, Pittsburgh, PA (US)

Correspondence Address: STEINBERG & RASKIN, P.C., 1140 AVENUE OF THE AMERICAS, 15th FLOOR, NEW YORK, NY, 10036-5803, US

(21) Appl. No.: 10/972,939

(22) Filed: Oct. 25, 2004

(51) Int. Cl. A61K 31/7052

(52) U.S. Cl. 514/43

(57) ABSTRACT

Methods for treatment and prevention of bacterial vaginosis are described. In order to treat a patient diagnosed with bacterial vaginosis, an antibiotic is administered to the patient and all infected participant-partners. Bacterial vaginosis is prevented in a human patient planning to participate in activities in which the patient's vagina may be contacted directly or indirectly by oral flora from a participant by cleansing the mouths of the patient and the patient's partner with an oral antiseptic prior to engaging in the activities.

### BACKGROUND OF THE INVENTION

1. Field of Invention

This invention relates in general to a method to treat women suffering from bacterial vaginosis and to prevent the occurrence of bacterial vaginosis in women who plan to participate in activities by which the patient's vagina may be contaminated by oral flora.

2. Description of the Prior Art

Bacterial vaginosis is the most common vaginal disease in women of childbearing age, accounting for fifty percent of vaginal infections. Bacterial vaginosis may be either symptomatic or asymptomatic, with fifty percent of cases being asymptomatic. When symptomatic, bacterial vaginosis is associated with a vaginal discharge that may or may not have an odor. Although many studies have focused on isolating the causative bacterium of bacterial vaginosis, no one bacterium has ever been isolated as the sole etiologic agent of the disease. Currently it is believed that bacterial vaginosis is caused by a change in the vaginal flora with a loss of Lactobacilli, an increase in vaginal pH, and an increase in multiple anaerobic flora. Thus there is not one specific bacterium that is responsible for bacterial vaginosis; rather it is of a polymicrobial etiology.

Bacterial vaginosis is best diagnosed by viewing cells under a microscope. A wet mount of the cells is prepared by smearing some of the vaginal discharge on a slide and adding normal saline. The slide is viewed under a microscope at 400× power. The presence of clue cells is diagnostic of bacterial vaginosis. Clue cells are epithelial cells that are so extensively coated by cocci bacteria that the cell membranes of the epithelial cells are obstructed from view.

The importance of bacterial vaginosis lies in both its symptomatology and its associated sequelae. Bacterial vaginosis can be associated with pelvic inflammatory disease, endometritis, and vaginal cuff cellulitis. Chorioamnionitis and neonatal sepsis have also been reported as associated with bacterial vaginosis. Furthermore, over the years, a strong correlation between bacterial vaginosis and adverse pregnancy outcomes (e.g., preterm labor, preterm birth, premature rupture of the membranes, chorioamnionitis) has been shown. The Center for Disease Control (“CDC”) recommends treatment of all symptomatic pregnant women to prevent these adverse outcomes. Specialists also recommend the screening and treatment of asymptomatic pregnant women at high-risk for preterm delivery (i.e., those with a previous history of preterm delivery).

While the association between bacterial vaginosis and pre-term delivery is well-established, the optimal treatment regimen poses some concerns. Studies have been conducted using various treatment regimens, among which are oral and/or intravaginal metronidazole as well as oral and/or intravaginal clindamycin.

Metronidazole is an antibacterial and antiprotozoal agent used in the treatment of various infections including bacterial vaginosis. It is a nitroimidazole antibiotic that acts as an electron acceptor in the metabolism of the bacteria, causing growth disturbances in the susceptible microorganism. Metronidazole can be administered either parenterally (oral and intravenous) or topically (intravaginal).

One study showed that while clindamycin vaginal cream is an effective treatment for bacterial vaginosis, intravaginal treatment has not been shown to be effective in eradicating microorganisms in the upper genital tract. Numerous studies have shown that metronidazole is an effective treatment of bacterial vaginosis in pregnant women, thereby preventing preterm delivery. A double-blind study by Morales et al. shows that high-risk women (i.e., women with previous history of preterm delivery) treated with oral metronidazole had fewer hospital admissions for preterm labor, preterm births, PROM (premature rupture of the membranes), and low-birth-weight infants. Furthermore, two separate studies found that oral metronidazole treatment was effective in long-term suppression of bacterial vaginosis flora for 2-3 months in pregnant women. There is limited data concerning the use of metronidazole vaginal gel (Metrogel®) during pregnancy. Therefore, systemic therapy may be required to eradicate upper tract infection in order to reduce preterm delivery.

Since concerns were raised regarding the possible teratogenic and mutagenic effect of metronidazole in pregnancy, studies were conducted in the hope of addressing these concerns. Among the concerns were the risk of prenatal exposure for neuroblastomas, midline facial defects, and leukemias. A cohort study done by Piper et al. showed no evidence that prenatal use of metronidazole increases the risk of overall birth defect occurrence. A retrospective cohort study of children showed no increase in the risk of cancers associated with in utero exposure to metronidazole. While a non-significant association was found with neuroblastoma, this requires further evaluation.

Investigators have examined several risk factors for the development and transmission of bacterial vaginosis, including the use of the intrauterine device, vaginal douching, hormonal alterations, and race. Most pertinent to the present invention are the studies that have been performed in an attempt to identify whether bacterial vaginosis is transmitted from one individual to another. Bacterial vaginosis has been demonstrated to be more prevalent in individuals who are sexually active than those who are not sexually active. Support for sexual activity as a mode of transmission includes a correlation between new sexual partners and the number of lifetime sexual partners to the presence of bacterial vaginosis, a decreased rate of bacterial vaginosis in monogamous couples, and a decreased incidence of bacterial vaginosis in virgins, where “virgin” was defined as a female who had not had sexual intercourse. Studies have also demonstrated the presence of bacteria associated with bacterial vaginosis in the urine and urethra of males whose partners were recently diagnosed with bacterial vaginosis. However, these bacteria did not persist in the male subjects after two weeks of condom use. The lack of persistence of these bacteria in the male urethra and/or urine has been argued to demonstrate a lack of sexual transmission for bacterial vaginosis. Further studies in males whose partners were diagnosed with bacterial vaginosis have demonstrated that males may or may not be colonized with bacteria associated with bacterial vaginosis. In addition, males are not symptomatic. A further argument against sexual activity as a route of transmission of bacterial vaginosis is that the routine treatment of males has not been demonstrated to be beneficial in preventing recurrence in females.

Important to the present invention is the fact that, in regard to sexual activity, the prior art has focused on sexual activity as defined by vaginal penetration with a penis. In contrast, the present invention focuses on the transmission of bacterial vaginosis by oral sexual relations or contact, wherein oral sex is defined as the direct or indirect contact of the vagina with oral flora.

The present investigation is based on the discovery that bacterial vaginosis is transmitted during oral sex by oral flora, which is the population of microorganisms contained in the mouth. Specific microorganisms will colonize a particular tissue surface depending on the ability of the organism to attach to the host surface. Thus, different areas of the mouth have their own unique microbial population. At any time, the number of microorganisms present in the saliva can range up to 10⁹ organisms per milliliter. Despite the presence of such a multitude of microorganisms in the mouth, acute oral infections are not common because the combination of the microorganisms, immunoglobins, complement factors, and other phagocytic cells contribute to the normal host defense mechanisms.

Given the prevalence and symptomatology of bacterial vaginosis, there is a need for a method that not only treats, but also prevents, the disease. Accordingly, it is an object of the invention to provide not only a method of treating bacterial vaginosis but also to provide an effective method for its prevention.

### SUMMARY AND OBJECTS OF THE INVENTION

The present invention is based on the discovery that there is a relationship between oral flora and bacterial vaginosis. The study found that individuals who engage in oral sexual relations have an increased incidence of bacterial vaginosis in comparison to individuals who do not engage in oral sexual relations. Based on this study it has been concluded that oral flora subsequently transferred to the vagina is the principal cause of bacterial vaginosis.

According to the method of prevention of the claimed invention, the mouth of each participant planning to participate in activities by which the patient's vagina may be contacted by oral flora, directly or indirectly, is cleansed with an effective dose of oral antiseptic not more than four to six hours before the activity, wherein the activity occurs only after the cleansing and prior to any contact by additional sources of oral flora, and wherein the indirect contact includes, for example, auto-inoculation by the patient having oral contact with a partner after cleansing by the partner.

According to the method of treatment and prevention, an effective amount of a compound to treat bacterial vaginosis is administered to a patient infected with bacterial vaginosis, and the mouth of at least one participant planning to participate in activities by which the patient's vagina may be contacted by oral flora, directly or indirectly, is cleansed with an oral antiseptic not more than four to six hours before participating in the activity.

It is therefore an object of the present invention to provide a method to prevent the development of bacterial vaginosis in women who participate in activities by which the patient's vagina may be contacted directly or indirectly by oral flora.

It is also an object of the present invention to provide a method to treat bacterial vaginosis.

It is also an object of the present invention to provide not only a method to treat bacterial vaginosis, but also a method to prevent bacterial vaginosis.

While the foregoing has been set forth in considerable detail, it is to be understood that the detailed embodiments are presented for elucidation and not limitation. Variations may be made but are within the principles of the invention.

### DETAILED DESCRIPTION OF THE PREFERRED EMBODIMENT

Oral flora are the population of bacteria that naturally exist in the mouth. When oral flora contact the vagina, bacterial vaginosis usually develops. Therefore, patients who participate in oral sexual relations, or any act in which the vagina may be contacted by oral flora, are exposed to a risk of developing bacterial vaginosis. This contact can occur directly through contact between the patient's vagina and the mouth, tongue, lips, or oral cavity of the patient's partner. Contact may also occur indirectly when the oral flora contact an object, and that object subsequently comes into contact with the patient's vagina. Contact may also occur by auto-inoculation, where oral flora originating in the patient's mouth indirectly infect that patient's vagina through contact of the oral flora with an object that subsequently comes into contact with the patient's vagina. Such objects include, but are not limited to, the partner's mouth, penis or a sex toy.

The present invention provides a method for the treatment and prevention of bacterial vaginosis for patients planning to participate in activities by which the patient's vagina may be either directly or indirectly contacted by oral flora. According to the method of prevention of the claimed invention, the mouth of each participant, including preferably the patient, is cleansed with an effective dose of an oral antiseptic, preferably about 30 cc or 1 ounce for thirty seconds. In the preferred embodiment, the cleansing occurs immediately prior to the participants engaging in activities leading to contact of the oral flora with the patient's vagina, but in other embodiments the cleansing may occur not more than four to six hours prior to the activities. The act must occur after the cleansing, but prior to any intervention by any additional sources of contact of the vagina with the oral flora, where intervention is defined as the subsequent contact of the mouth of the patient or the participant with oral flora after the mouths have been cleansed.

In the preferred embodiment, the mouths of the participants are cleansed with an oral antiseptic, for example, a commercial antibacterial mouthwash such as Scope® or Listerine®. In another embodiment of the invention, the oral antiseptic is a prescription antibacterial mouthwash such as Peridex® (chlorhexidine gluconate). In still another embodiment of the invention, the oral antiseptic is an alcoholic beverage consumed by the participants prior to the act.

According to the method of treatment of the present invention, a patient diagnosed with bacterial vaginosis is administered an effective amount of an antibiotic to treat the infection. Effective treatment methods are administration of either metronidazole or clindamycin. In the preferred embodiment, the method of treatment is Flagyl® (500 mg metronidazole, orally) administered to the patient twice daily for seven days. In another embodiment, bacterial vaginosis is treated intravaginally using either Metrogel® (metronidazole vaginal gel) for five days or Cleocin® (clindamycin vaginal cream) for seven days. In the preferred embodiment, the patient undergoing the method of treatment is advised not to participate in activities by which the patient's vagina may be either directly or indirectly contacted by oral flora for at least three days, and preferably seven days, following the initiation of administration of the antibiotic.

Furthermore, in the preferred embodiment, the method of treatment also comprises administering to an infected participant-partner of the infected patient an effective amount of an antibiotic, where the infected participant-partner is defined as all sexual partners of either the infected patient while the patient was infected with bacterial vaginosis or other partners similarly infected with bacterial vaginosis. The method of treatment of a male participant-partner of the infected patient is Flagyl® (500 mg metronidazole, orally) administered to the participant-partner twice daily for seven days. The method of treatment of a female participant-partner of the infected patient may be either Flagyl® (500 mg metronidazole, orally) twice daily for seven days, Metrogel® (metronidazole vaginal gel) for five days, or Cleocin® (clindamycin vaginal cream) for seven days.

According to the method of treatment and prevention, a patient who is diagnosed with bacterial vaginosis is treated and then educated on the method of prevention.

It should be understood that according to the present invention each participant need not perform the oral sexual activity per se, but may rather be solely the recipient of oral sexual activity. In such situations, at least the participant performing the oral sexual activity on other participant(s) can practice the claimed method and receive the benefits thereof.

Those skilled in the art will realize that such changes or modifications of the invention or combinations of the elements, variations, equivalents, or improvements therein are still within the scope of the invention as defined in the appended claims.

What is claimed is:

1. A method to prevent bacterial vaginosis in a patient planning to participate in activities in which said patient's vagina may be contacted by oral flora from a participant, said method comprising at least one participant cleansing an oral region with an effective dose of an oral antiseptic or alcoholic beverage not more than 4 to 6 hours before said activity, wherein said activity occurs after said cleansing and prior to any contact with oral flora from a non-cleansed participant.

2. A method to treat and prevent bacterial vaginosis in a patient planning to participate in activities in which said patient's vagina may be contacted by oral flora from a participant, said method comprising the steps of:
a. administering to said patient infected with bacterial vaginosis an effective amount of a compound selected from the group consisting of metronidazole and clindamycin, and
b. cleansing an oral region of at least one participant with an effective dose of an oral antiseptic not more than 4 to 6 hours prior to engaging in said activity, wherein said activity occurs after said cleansing and prior to any contact with oral flora from a non-cleansed participant.

3. The method of claim 2 wherein an infected participant-partner is administered an effective amount of said compound.

4. The method of claim 2 wherein said compound is Flagyl®.

5. The method of claim 2 wherein said compound is Metrogel®.

6. The method of claim 2 wherein said compound is Cleocin®.

7. In a method of treating a patient infected with bacterial vaginosis comprising the step of administering to said infected patient an effective amount of a compound selected from the group consisting of metronidazole and clindamycin, wherein the improvement comprises the step of preventing bacterial vaginosis in said patient planning to participate in activities in which said patient's vagina may be contacted by oral flora from a participant by cleansing an oral region of at least one participant with an effective dose of an oral antiseptic not more than four to six hours prior to participating in said activity, wherein said activity occurs after said cleansing and prior to any contact by oral flora from a non-cleansed participant.

8. The method of claim 7 wherein said improvement further comprises administering to an infected participant-partner an effective amount of said compound.

9. The methods of claim 1, 2 or 7 wherein said contact is direct.

10. The methods of claim 1, 2 or 7 wherein said contact is indirect.

11. The methods of claim 1, 2 or 7 wherein said contact is auto-inoculation.

12. The methods of claim 1, 2 or 7 wherein said oral antiseptic is a commercial antibacterial mouthwash.

13. The methods of claim 1, 2 or 7 wherein said oral antiseptic is a prescription mouthwash.

14. The methods of claim 1, 2 or 7 wherein said oral antiseptic is an alcoholic beverage.

15. The methods of claim 1, 2 or 7 wherein said oral cleansing occurs for at least thirty seconds.

16. The methods of claim 2 or 7 wherein said patient is advised not to participate in said activities until at least three days following the initiation of said administration of said compound.
